(19) United States
(12) Plant Patent Application Publication
(10) Pub. No.: US 2026/0114346 P1
(43) Pub. Date: Apr. 23, 2026

(54) Digitalis Plant Named 'Balroxleam'

(50) ;

(71) Applicant: Ball Horticultural Company, West Chicago, IL (US)

(72) Inventor: Paul Gehrke, Miami, FL (US)

(21) Appl. No.: 19/356,380

(22) Filed: Oct. 13, 2025

Related U.S. Application Data
(60) Provisional application No. 63/709,358, filed on Oct. 18, 2024.

(51) Int. Cl. A01H 6/00; A01H 5/02

(52) U.S. Cl. CPC A01H 6/00; A01H 5/02
USPC PLT/263.1

(57) ABSTRACT

A new and distinct cultivar of Digitalis plant named ‘Balroxleam’, characterized by its light-yellow colored flowers, dark, green-colored foliage, and moderately vigorous, columnar-upright growth habit, is disclosed.

Latin name of genus and species of plant claimed: Digitalis hybrida.

Variety denomination: ‘Balroxleam’.

### BACKGROUND OF THE INVENTION

The present invention relates to a new and distinct cultivar of Digitalis plant botanically known as Digitalis hybrida and hereinafter referred to by the cultivar name ‘Balroxleam’.

The new cultivar is a naturally occurring whole plant mutation of Digitalis hybrida Arctic Fox Rose ‘Balroxose’, U.S. Plant Pat. No. 31,900. The new Digitalis plant was discovered and selected by the inventor as a single flowering plant from within a population on plants of ‘Balroxose’ in a controlled greenhouse environment in Miami, Florida during the spring of 2020.

Asexual reproduction of the new cultivar by terminal stem cuttings since the spring of 2020 in West Chicago, Illinois has demonstrated that the new cultivar reproduces true to type with all the characteristics, as herein described, firmly fixed and retained through successive generations of such asexual propagation.

### SUMMARY OF THE INVENTION

The following characteristics of the new cultivar have been repeatedly observed and can be used to distinguish ‘Balroxleam’ as a new and distinct cultivar of Digitalis plant:
1. Light-yellow colored flowers;
2. Dark, green-colored foliage; and
3. Moderately vigorous, columnar-upright growth habit.

Plants of the new cultivar differ from plants of the sport parent primarily in having light-yellow colored flowers.

Of the many commercially available Digitalis cultivars, the most similar in comparison to the new cultivar is ‘Waldigone’, U.S. Plant Pat. No. 20,937. However, in side-by-side comparison, plants of the new cultivar differ from plants of ‘Waldigone’ in at least the following characteristics:

1. Plants of the new cultivar have flowers with a more light-yellow color than plants of ‘Waldigone’;

2. Plants of the new cultivar have flowers with deeper corollas than plants of ‘Waldigone’.

### BRIEF DESCRIPTION OF THE PHOTOGRAPHS

The accompanying photographs show, as nearly true as it is reasonably possible to make the same in color illustrations of this type, typical flower and foliage characteristics of the new cultivar. Colors in the photographs may differ slightly from the color values cited in the detailed description, which accurately describes the colors of ‘Balroxleam’. The plants were approximately 3.5 months old and grown in 4-in containers for approximately 12 weeks in a double polyethylene greenhouse in West Chicago, Illinois.

FIG. 1 illustrates a side view of the overall growth and flowering habit of ‘Balroxleam’.

FIG. 2 illustrates a close-up view of an individual inflorescence of ‘Balroxleam’.

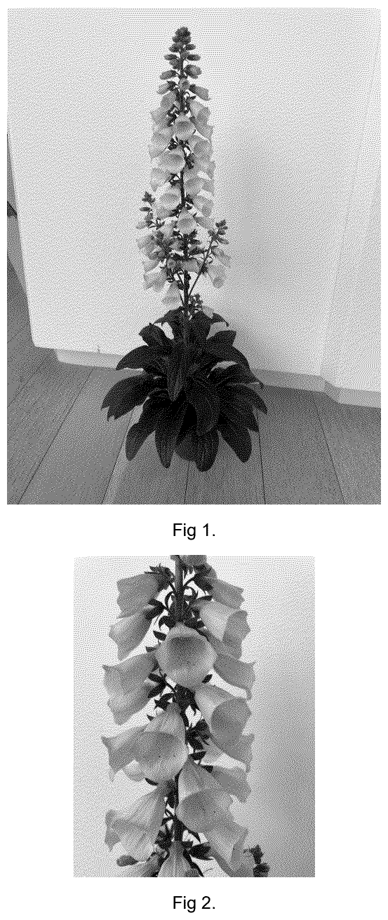

### DETAILED BOTANICAL DESCRIPTION

The new cultivar has not been observed under all possible environmental conditions to date. Accordingly, it is possible that the phenotype may vary somewhat with variations in the environment, such as temperature, light intensity, and day length, without, however, any variance in genotype.

The chart used in the identification of colors described herein is The R.H.S. Colour Chart of The Royal Horticultural Society, London, England, 2015 edition, except where general color terms of ordinary significance are used. The color values were determined in February 2025 under natural light conditions in West Chicago, Illinois.

The following descriptions and measurements describe approximately 3.5-month-old plants produced from cuttings from stock plants and grown in a double polyethylene greenhouse under conditions comparable to those used in commercial practice. The plants were grown in West Chicago, Illinois in 4-inch pots for 5 weeks utilizing a soilless growth medium. Plants were not pinched prior to transplant. After approximately five weeks, plants were then transplanted and grown for approximately eight weeks. Greenhouse temperatures were maintained at approximately 60° F. to 65° F. (15.5° C. to 18.3° C.) during the day and approximately 55° F. to 60° F. (12.8° C. to 15.5° C.) during the night. Supplemental lighting was used. Measurements and numerical values represent averages of typical plants.
Botanical classification: Digitalis hybrid ‘Balroxleam’.
Parentage:
   Parent.—Digitalis hybrida Arctic Fox Rose ‘Balroxose’, U.S. Plant Pat. No. 31,900.
Propagation:
   Type cutting.—Terminal stem.
   Time to initiate roots.—Approximately 15 to 20 days.
   Time to produce a rooted cutting.—Approximately 42 to 49 days.
   Root description.—Fine, fibrous.
   Rooting habit.—Freely branching.
Plant description:
   Commercial crop time.—Approximately 12 to 14 weeks from a rooted cutting to finish in a 15 cm pot, vernalization not required.
   Growth habit and general appearance.—Herbaceous perennial, moderately vigorous, columnar-upright.
   Hardiness.—Hardiness — USDA Zone 5a (−20° F. to −15° F./−28.9° C. to −26° C.).
   Size.—Height from soil level to top of plant plane: Approximately 44 cm. Width: Approximately 21 cm.
   Branching habit.—Freely branching. Quantity of branches per plant: Approximately 1 main branch, with approximately 4 basal clumps.
   Main branch.—Strength: Strong. Aspect: Erect. Length to base of inflorescence: Approximately 12.0 cm. Diameter: Approximately 0.5 cm. Length of central internode: Approximately 4.0 cm. Texture: Densely pubescent. Gland color: Colorless, transparent. Color of young stem: 143B. Color of mature stem 144A.
Foliage description:
   General Description.—Quantity of leaves per main branch: Approximately 11. Fragrance: None. Form: Simple. Arrangement: Rosette at plant base and dextrorse on flowering stems.
   Leaves.—Aspect: Perpendicular or obtuse angle to stem. Shape: Elliptic to narrow elliptic. Margin: Serrate. Apex: Acute. Base: Attenuate, cauline leaves sessile. Venation pattern: Pinnate. Length of mature leaf: Approximately 11.0 cm. Width of mature leaf: Approximately 3.0 cm. Texture of upper surface: Glabrous. Texture of lower surface: Densely pubescent on venation. Color of upper surface of young foliage: 137A with venation of 146C. Color of lower surface of young and mature foliage: Closest to 138B with venation of 146D and midvein of closest to 145D. Color of upper surface of mature foliage: NN137A with venation of 146C.
   Petiole.—Length: Approximately 2.0 cm. Diameter: Approximately 9.0 mm. Texture: Densely pubescent. Color: Closest to 138B.
Flowering description:
   Flowering habit.—‘Balroxleam’ is a long-day facultative, freely flowering under outdoor growing conditions, blooming from April through September.
   Lastingness of individual flower on the plant.—Approximately 10 days.
Inflorescence description:
   General Description.—Type: Terminal raceme. Aspect: Primarily erect. Quantity per plant: Approximately 12. Fragrance: None. Length or height: Approximately 28.0 cm. Width: Approximately 8.0 cm. Quantity of fully open flowers per inflorescence: Approximately 12.
   Peduncle.—Strength: Strong. Aspect: Erect. Length: Approximately 19.0 cm. Diameter: Approximately 4.0 mm to 8.0 mm. Texture: Densely glandular pubescent. Gland color: Colorless, transparent. Color: 137B.
Flower description:
   Type.—Single, campanulate, not persistent.
   Bud just before opening.—Shape: Ovoid. Length: Approximately 1.3 cm. Diameter: Approximately 7.0 mm. Color: Calyx of 139C, petal portion 144C.
   Corolla.—Shape: Bilabiate. Aspect: Facing outward and downward. Length: Approximately 3.7 cm. Width: Approximately 2.5 cm. Depth: Approximately 3.5 cm.
   Petals.—Quantity: 5 petals fused at base forming a throat and consisting of an upper lip with three lobes and a single-lobed projecting lower lip. Shape: Obovate. Margin: Entire, ciliate. Apex: Obtuse, with lateral lobes notched and lower lobe slightly rolled under.
   Upper lip.—Length of upper lobe from throat: Approximately 3.0 mm. Width of upper lobe: Approximately 2 cm. Length of lateral lobes from throat: Approximately 4.0 mm. Width of lateral lobes: Approximately 1.1 cm. Texture of upper (inner) surface: Glabrous. Texture of lower (outer) surface: Sparsely glandular pubescent. Gland color: Colorless, transparent. Color of upper (inner) surface when fully open: 4D. Color of lower (outer) surface when fully open: 2D.
   Lower lip.—Length of lobe from throat: Approximately 8.0 mm. Width: Approximately 1.8 cm. Texture of upper (inner) surface: Sparsely pubescent. Texture of lower (outer) surface: Moderately glandular pubescent. Gland color: Colorless, transparent Color of upper (inner) surface when fully open: 4D. Color of lower (outer) surface when fully open: 2D.
   Corolla tube.—Length: Approximately 1.5 cm. Width: Approximately 0.7 cm. Texture of inner surface: Sparsely pubescent on lower portion (nectar glide). Texture of outer surface: Glabrous. Color of inner surface: 4D with spots of N45 forming nectar glide beginning at lower lip. Color of outer surface: 2D.
   Calyx.—Shape: Cupped, tips flare out with age. Diameter: Approximately 2.0 cm.
   Sepals.—Quantity per flower: 5, imbricate. Shape: Lanceolate. Apex: Acute. Base: Truncate. Length: Approximately 1.1 cm. Width: Approximately 5.0 mm. Texture of upper and lower surfaces: Densely pubescent. Color of upper (inner) and lower (outer) surfaces: 137B.
   Pedicel.—Strength: Strong. Aspect: Acute angle to stem. Length: Approximately 6.0 mm. Diameter: Approximately 1.0 mm. Texture: Densely glandular pubescent. Gland color: Colorless, transparent. Color: 137C.
   Reproductive organs.—Androecium: Stamen quantity: 4, basifixed. Stamen length: Longer pair at approximately 2.0 cm and shorter pair at 1.4 cm. Filament length of fixed portion: Approximately 1.1 cm. Filament color: 1D. Anther shape: Reniform. Anther length: Approximately 2.0 mm. Anther color: 154C. Pollen amount: Sparce. Pollen color: 155D. Gynoecium: Pistil quantity: 1 per flower. Pistil length: Approximately 1.6 cm. Stigma shape: Bifid. Stigma length: Approximately 1.0 mm. Stigma color: 155D. Style length: Approximately 1.4 cm. Style color: NN155A. Ovary length: Approximately 8.0 mm. Ovary texture: Densely pubescent. Ovary color: 143C.
Seed and fruit production: Neither seed nor fruit production has been observed.
Disease and pest resistance: Resistance to pathogens and pests common to Digitalis has not been observed.

What is claimed is:

1. A new and distinct cultivar of Digitalis plant named ‘Balroxleam’, substantially as herein illustrated and described.
